(19) United States
(12) Patent Application Publication
(10) Pub. No.: US 2019/0111096 A1
(43) Pub. Date: Apr. 18, 2019

(54) USES OF MENTHA ESSENTIAL OIL

(71) Applicant: TCI Co., Ltd., Taipei (TW)

(72) Inventor: Yung-Hsiang LIN, Taipei (TW)

(21) Appl. No.: 16/158,399

(22) Filed: Oct. 12, 2018

Related U.S. Application Data
(60) Provisional application No. 62/573,929, filed on Oct. 18, 2017.

(30) Foreign Application Priority Data
Sep. 20, 2018 (TW) 107133214

(51) Int. Cl. A61K 36/534; A61P 29/00; A61P 35/00; A61P 25/28; A61P 25/20; A61P 3/10; A61P 3/04; A61P 9/00

(52) U.S. Cl. CPC A61K 36/534; A61P 29/00; A61P 35/00; A61P 25/28; A61K 9/0053; A61P 3/10; A61P 3/04; A61P 9/00; A61P 25/20

(57) ABSTRACT

A method for at least one of inhibiting obesity, preventing metabolic syndrome associated with obesity, treating neurodegenerative disease, preventing neurodegenerative disease, treating cardiovascular disease, preventing cardiovascular disease, treating type 2 diabetes, preventing type 2 diabetes, ameliorating sleep disturbance, regulating sleep, helping fall asleep, inhibiting inflammation, treating cancer and preventing cancer is provided, wherein the method comprises administering to a subject in need an effective amount of Mentha essential oil. A method for helping reduce the production of free radical is also provided, wherein the method comprises administering to a subject in need an effective amount of Mentha essential oil.

### CROSS REFERENCE TO RELATED APPLICATIONS

This application claims priority to U.S. Provisional Application Ser. No. 62/573,929 filed on Oct. 18, 2017, and to Taiwan Patent Application No. 107133214 filed on Sep. 20, 2018, the disclosures of which are incorporated herein in their entirety by reference.

### FIELD OF THE INVENTION

The present invention relates to the use of Mentha essential oil, including the use of Mentha essential oil in inhibiting obesity, preventing metabolic syndrome associated with obesity, treating neurodegenerative disease, preventing neurodegenerative disease, treating cardiovascular disease, preventing cardiovascular disease, treating type 2 diabetes, preventing type 2 diabetes, ameliorating sleep disturbance, regulating sleep, helping fall asleep, inhibiting inflammation, treating cancer and/or preventing cancer. The present invention also relates to the use of Mentha essential oil in helping reduce the production of free radical. The present invention also relates to the use of Mentha essential oil in increasing expression of SOD1 gene, increasing expression of CAT gene, increasing expression of UCP2 gene, increasing expression of CRY gene, increasing expression of PER2 gene, decreasing expression of IL-8 gene and/or decreasing expression of IL-1β gene.

### BACKGROUND OF THE INVENTION

Essential oil is an oil product containing aromatic compound(s) and is extracted from plants. Specifically, essential oil can be provided by extracting the flowers, leaves, roots, seeds, peels, fruits and/or stems of plant(s) by such as distillation, expeller process, solvent extraction, enfleurage, and supercritical fluid extraction (SFE). Depending on the species and parts of plant(s) from which the essential oil is produced, the essential oil may have different effects and can be used in different forms in the daily life or pharmaceutical industry as well.

Mentha, a perennial plant, is a creeping plant and has leaves smelling cool and fresh and flowers in light purple color. Mentha has a long history of use and is usually used as an additive in food or beverages or as a fragrance. In general, Mentha essential oil is obtained by extracting the stems, leaves and/or flowers of Mentha via distillation. Mentha essential oil smells cool and fresh and thus is often used for refreshing oneself, clearing the smell of mouth and/or ameliorating headache. Furthermore, Mentha essential oil is effective in sterilization, softening skin and helping digestion, and thus is widely applied in daily life.

Aside from the above uses, inventors of the present invention discovered that Mentha essential oil is effective in regulating the expressions of genes, i.e., SOD1, CAT, UCP2, CRY, PER2, IL-8 and IL-1β, and thus can be used for treating, preventing and/or regulating diseases or physiological functions related to the above genes.

### SUMMARY OF THE INVENTION

An objective of the present invention is to provide a use of Mentha essential oil in the manufacture of a pharmaceutical composition, wherein the pharmaceutical composition is for at least one of inhibiting obesity, preventing metabolic syndrome associated with obesity, treating neurodegenerative disease (including amyotrophic lateral sclerosis), preventing neurodegenerative disease, treating cardiovascular disease (including atherosclerosis), preventing cardiovascular disease, treating type 2 diabetes, preventing type 2 diabetes, ameliorating sleep disturbance, regulating sleep, inhibiting inflammation, treating cancer (including lung cancer, melanoma and colon cancer) and preventing cancer. Preferably, the pharmaceutical composition is provided in a form for oral administration, inhalation administration or transdermal administration.

Another objective of the present invention is to provide a use of Mentha essential oil in at least one of helping fall asleep, helping reduce the production of free radical and making body fat be difficult to form. Preferably, the Mentha essential oil is taken through oral, inhalation, or transdermal route.

Still another objective of the present invention is to provide a use of Mentha essential oil in the manufacture of a pharmaceutical composition, wherein the pharmaceutical composition is used for at least one of increasing expression of SOD1 gene, increasing expression of CAT gene, increasing expression of UCP2 gene, increasing expression of CRY gene, increasing expression of PER2 gene, decreasing expression of IL-8 gene and decreasing expression of IL-1β gene. Preferably, the pharmaceutical composition is provided in a form for oral administration, inhalation administration or transdermal administration.

Yet another objective of the present invention is to provide a pharmaceutical composition for inhibiting obesity, preventing metabolic syndrome associated with obesity, treating neurodegenerative disease (including amyotrophic lateral sclerosis), preventing neurodegenerative disease, treating cardiovascular disease (including atherosclerosis), preventing cardiovascular disease, treating type 2 diabetes, preventing type 2 diabetes, ameliorating sleep disturbance, regulating sleep, inhibiting inflammation, treating cancer (including lung cancer, melanoma and colon cancer), and/or preventing cancer. The pharmaceutical composition comprises an effective amount of Mentha essential oil. Preferably, the pharmaceutical composition is provided in a form for oral administration, inhalation administration or transdermal administration.

Yet another objective of the present invention is to provide a care product composition for helping fall asleep, helping reduce the production of free radical, and/or making body fat be difficult to form. The care product composition comprises an effective amount of Mentha essential oil. Preferably, the care product composition is provided in a form for oral administration, inhalation administration or transdermal administration.

Yet another objective of the present invention is to provide a pharmaceutical composition for increasing expression of SOD1 gene, increasing expression of CAT gene, increasing expression of UCP2 gene, increasing expression of CRY gene, increasing expression of PER2 gene, decreasing expression of IL-8 gene, and/or decreasing expression of IL-1β gene. The pharmaceutical composition comprises an effective amount of Mentha essential oil. Preferably, the pharmaceutical composition is provided in a form for oral administration, inhalation administration or transdermal administration.

Yet another objective of the present invention is to provide a method for at least one of inhibiting obesity (including making body fat be difficult to form), preventing metabolic syndrome associated with obesity, treating neurodegenerative disease (including amyotrophic lateral sclerosis), preventing neurodegenerative disease, treating cardiovascular disease (including atherosclerosis), preventing cardiovascular disease, treating type 2 diabetes, preventing type 2 diabetes, ameliorating sleep disturbance, regulating sleep, helping fall asleep, inhibiting inflammation, treating cancer (including lung cancer, melanoma and colon cancer) and preventing cancer. The method comprises administering to a subject in need an effective amount of Mentha essential oil. In the method according to the present invention, the Mentha essential oil can be administered as the pharmaceutical composition described above.

Yet another objective of the present invention is to provide a method for helping reduce the production of free radical. The method comprises administering to a subject in need an effective amount of Mentha essential oil. In the method according to the present invention, the Mentha essential oil can be administered as the care product composition described above.

Yet another objective of the present invention is to provide a method for at least one of increasing expression of SOD1 gene, increasing expression of CAT gene, increasing expression of UCP2 gene, increasing expression of CRY gene, increasing expression of PER2 gene, decreasing expression of IL-8 gene and decreasing expression of IL-1β gene. The method comprises administering to a subject in need an effective amount of Mentha essential oil. In the method according to the present invention, the Mentha essential oil can be administered as the pharmaceutical composition described above.

The detailed technology and preferred embodiments implemented for the present invention are described in the following paragraphs accompanying the appended drawings for people skilled in this field to well appreciate the features of the claimed invention.

### BRIEF DESCRIPTION OF THE DRAWINGS

FIG. 1 shows the influence of Mentha essential oil on the expression of SOD1 gene of peripheral blood mononuclear cells (PBMCs), wherein the result of “W0” group refers to the expression level before the administration of Mentha essential oil, and the results of “W1” and “W2” groups refer to the expression level after the continuous administration of Mentha essential oil for one week and two weeks, respectively (** represents the result is significantly different from that of the “W0” group, p<0.01);

FIG. 2 shows the influence of Mentha essential oil on the expression of CAT gene in PBMCs of the “W0”, “W1” and “W2” groups;

FIG. 3 shows the influence of Mentha essential oil on the expression of UCP2 gene in PBMCs of the “W0”, “W1” and “W2” groups (* represents the result is significantly different from that of the “W0” group, p<0.05);

FIG. 4 shows the influence of Mentha essential oil on the expression of CRY gene in PBMCs of the “W0”, “W1” and “W2” groups (** represents the result is significantly different from that of the “W0” group, p<0.01);

FIG. 5 shows the influence of Mentha essential oil on the expression of PER2 gene in PBMCs of the “W0”, “W1” and “W2” groups (** represents the result is significantly different from that of the “W0” group, p<0.01);

FIG. 6 shows the influence of Mentha essential oil on the expression of IL-8 gene in PBMCs of the “W0”, “W1” and “W2” groups (*** represents the result is significantly different from that of the “W0” group, p<0.001); and

FIG. 7 shows the influence of Mentha essential oil on the expression of IL-1β gene in PBMCs of the “W0”, “W1” and “W2” groups (*** represents the result is significantly different from that of the “W0” group, p<0.001).

### DESCRIPTION OF THE PREFERRED EMBODIMENTS

The following will describe some of the embodiments of the present invention in detail. However, without departing from the spirit of the present invention, the present invention may be embodied in various embodiments and should not be limited to the embodiments described in the specification or defined in the appended claims.

Unless otherwise indicated herein, the expressions “a,” “an,” “the,” or the like recited in the specification of the present invention (especially in the claims) are intended to include both the singular and plural forms. The term “treat” or “treating” recited in this specification should not be construed as treating a subject until the subject is completely recovered, but should include maintaining the progression or symptoms of the diseases in a substantially static state, increasing the recovery rate of a subject, alleviating the severity of a particular condition of illness, or increasing the life quality of patients. The term “prevent” or “preventing” recited in this specification refers to inhibiting or preventing a particular condition of illness from breaking out, or maintaining good health in a sensitive subject to tolerate diseases. The term “regulate” or “regulating” recited in this specification refers to upregulating (includes inducing, stimulating, and enhancing) or downregulating (includes inhibiting and weakening) the physiological functions in a subject toward a normal state. The term “subject” recited in this specification refers to a mammalian, including human and non-human animals.

It was revealed by researches that an increment in the expression level of SOD1 gene is beneficial to the reduction of free radical production. Therefore, if the expression of SOD1 gene can be increased effectively, the effect of helping production of free radical can be provided. Researches also revealed that the deficiency of SOD1 involved in the occurrence of amyotrophic lateral sclerosis, and these can be noted in “Rachele A. Saccon et al., 2013, Is SOD1 loss of function involved in amyotrophic lateral sclerosis?” which is entirely incorporated hereinto by reference. Therefore, if the expression of SOD1 gene can be increased effectively, the effects of treating amyotrophic lateral sclerosis and preventing amyotrophic lateral sclerosis could be provided.

It was revealed by researches that an increment in the expression level of CAT gene is beneficial to the reduction of free radical production, while a decrement in the expression level of CAT gene may lead to type 2 diabetes, and these can be noted in “Santosh Sinha, 2005, Anti-oxidant gene expression imbalance, aging and Down syndrome” and “L'ASZL'O G'OTH, 2008, Catalase deficiency and type 2 diabetes,” which are entirely incorporated hereinto by reference. Therefore, if the expression of CAT gene can be increased effectively, the following effects could be provided: helping production of free radical, treating type 2 diabetes, and preventing type 2 diabetes.

It was revealed by researches that an increment in the expression level of UCP2 gene could increase the metabolic rate of brown fat and promote the consumption of fat to produce energy as well, and these can be noted in “Antonio Vidal-Puig et al., 1999, Effects of Obesity and Stable Weight Reduction on UCP2 and UCP3 Gene Expression in Humans,” which is entirely incorporated hereinto by reference. Therefore, if the expression of UCP2 gene can be increased effectively, the following effects could be provided: inhibiting obesity, preventing metabolic syndrome associated with obesity, and making body fat be difficult to form. Researches also revealed that an increment in the expression level of UCP2 gene can ameliorate the neurodegenerative disease and cardiovascular disease, and these can be noted in “Gustav Mattiasson et al., 2006, The Emerging Functions of UCP2 in Health, Disease, and Therapeutics,” which is entirely incorporated hereinto by reference. Therefore, if the expression of UCP2 gene can be increased effectively, the following effects could be provided: treating neurodegenerative disease, preventing neurodegenerative disease, treating cardiovascular disease, and preventing cardiovascular disease.

It was revealed by researches that an increment in the expression level of CRY gene could regulate the circadian rhythm in animal body, and these can be noted in “Beilin Zhang et al., 2016, Sleep Deprivation Influences Circadian Gene Expression in the Lateral Habenula,” which is entirely incorporated hereinto by reference. Therefore, if the expression of CRY gene can be increased effectively, the following effects could be provided: ameliorating sleep disturbance, regulating sleep, and helping fall asleep. Researches also revealed that an increment in the expression level of CRY gene could protects against the development of atherosclerosis, and these can be noted in “Lei Yang et al., 2015, Overexpression of CRY1 protects against the development of atherosclerosis via the TLR/NF-κB pathway,” which is entirely incorporated hereinto by reference. Therefore, if the expression of CRY gene can be increased effectively, the effect of treating atherosclerosis could be provided.

It was revealed by researches that an increment in the expression level of PER2 gene could regulate the circadian rhythm in animal body, and these can be noted in “Beilin Zhang et al., 2016, Sleep Deprivation Influences Circadian Gene Expression in the Lateral Habenula,” which is entirely incorporated hereinto by reference. Therefore, if the expression of PER2 gene can be increased effectively, the following effects could be provided: ameliorating sleep disturbance, regulating sleep, and helping fall asleep.

It was revealed by researches that a decrement in the expression level of IL-8 gene could lead to an inhibition of inflammation, and these can be noted in “Huibi CAO et al., 2005, Down-regulation of IL-8 expression in human airway epithelial cells through helper-dependent adenoviral-mediated RNA interference,” which is entirely incorporated hereinto by reference. Therefore, if the expression of IL-8 gene can be decreased effectively, the effect of inhibiting inflammation could be provided. Researches also revealed that an increment in the expression level of IL-8 gene may promote the growth of lung cancer cells, and these can be noted in “YM Zhu et al., 2004, Interleukin-8/CXCL8 is a growth factor for human lung cancer cells,” which is entirely incorporated hereinto by reference. Therefore, if the expression of IL-8 gene can be decreased effectively, the effects of treating lung cancer and preventing lung cancer could be provided.

It was revealed by researches that a decrement in the expression level of IL-1β gene could lead to an inhibition of inflammation. Researches also revealed that IL-1β gene has a significant expression in both the melanoma cells and colon cancer cells, and these can be noted in “Anne M Lewis et al., 2006, Interleukin-1 and cancer progression: the emerging role of interleukin-1 receptor antagonist as a novel therapeutic agent in cancer treatment,” which is entirely incorporated hereinto by reference. Therefore, if the expression of IL-1β gene can be decreased effectively, the following effects could be provided: treating melanoma, preventing melanoma, treating colon cancer and preventing colon cancer.

Inventors of the present invention incidentally discovered that Mentha essential oil can effectively increase the expression of SOD1 gene, increase expression of CAT gene, increase expression of UCP2 gene, increase expression of CRY gene, increase expression of PER2 gene, decrease expression of IL-8 gene and decrease expression of IL-1β gene. Therefore, the present invention relates to the use of Mentha essential oil, including: using Mentha essential oil in helping fall asleep, helping reduce the production of free radical and/or making body fat be difficult to form, using Mentha essential oil in manufacturing a pharmaceutical composition, providing a care product composition or a pharmaceutical composition comprising an effective amount of Mentha essential oil, and providing a method of administering to a subject in need an effective amount of the aforementioned care product composition or pharmaceutical composition. The care product composition provided in accordance with the present invention is for helping fall asleep, helping reduce the production of free radical and/or making body fat be difficult to form. The pharmaceutical composition provided in accordance with the present invention is for inhibiting obesity, preventing metabolic syndrome associated with obesity, treating neurodegenerative disease, preventing neurodegenerative disease, treating cardiovascular disease, preventing cardiovascular disease, treating type 2 diabetes, preventing type 2 diabetes, ameliorating sleep disturbance, regulating sleep, anti-inflammation, treating cancer and/or preventing cancer. In addition, the pharmaceutical composition and method provided in accordance with the present invention are also for increasing expression of SOD1 gene, increasing expression of CAT gene, increasing expression of UCP2 gene, increasing expression of CRY gene, increasing expression of PER2 gene, decreasing expression of IL-8 gene and/or decreasing expression of IL-1β gene.

The Mentha essential oil adopted in accordance with the present invention could be obtained from any suitable source. For example, the Mentha essential oil could be purchased from the market, and could also be obtained by extracting Mentha plant. The Mentha plants that could be used for proving the Mentha essential oil of the present invention include, but are not limited to, Mentha piperita, Mentha spicata, Mentha arvensis, and Mentha suaveolens.

The pharmaceutical composition provided in accordance with the present invention could be administered to a subject in need systemically or topically, and could be delivered by various drug delivery systems (DDSs), such as oral drug delivery system, transdermal drug delivery system, inhalation drug delivery system, etc. For example, to enhance bioavailability, control drug release speed, target the lesion precisely and reduce side effects, the pharmaceutical composition could be delivered by a liposome, a microcapsule, nanoparticles, microneedles, but is not limited thereby.

Depending on the desired purpose(s), the pharmaceutical composition provided in accordance with the present invention could be provided in any suitable form without particular limitations. For example, the pharmaceutical composition could be provided in a form suitable for administering to a subject in need by oral administration, inhalation administration or transdermal administration, but is not limited thereby. Depending on the desired purpose(s), the care product composition provided in accordance with the present invention could be administered to a subject in need systemically or topically, and could be provided in any suitable form without particular limitations. For example, the care product composition could be provided in a form for oral administration, inhalation administration, or transdermal administration, but is not limited thereby.

Depending on the form and purpose(s), a suitable carrier could be chosen and used to provide the pharmaceutical composition or care product composition. Examples of the carrier include excipients, diluents, auxiliaries, stabilizers, absorbent enhancers, disintegrating agents, hydrotropic agents, emulsifiers, antioxidants, adhesives, binders, tackifiers, dispersants, suspending agents, lubricants, hygroscopic agents, etc.

As a form for oral administration, the pharmaceutical composition of the present invention could comprise any pharmaceutically acceptable carrier that will not adversely affect the desired effects of the active ingredient (i.e., Mentha essential oil). Examples of suitable carrier include, but are not limited to, water, saline, dextrose, glycerol, ethanol or its analogs, oil (such as olive oil, castor oil, cottonseed oil, peanut oil, corn oil, germ oil), polyethylene glycol and combinations thereof. The pharmaceutical composition could be provided by any suitable method in any suitable form for oral administration, such as in the form of a capsule, a fluidextract, a solution, syrup, a suspension, a tincture, or an elixir, but is not limited thereby.

As a form for oral administration, the care product composition of the present invention could comprise any carrier that will not adversely affect the desired effects of the active ingredient (i.e., Mentha essential oil). Examples of suitable carriers include, but are not limited to, water, saline, dextrose, glycerol, ethanol or its analogs, oil (such as olive oil, castor oil, cottonseed oil, peanut oil, corn oil, germ oil), polyethylene glycol and combinations thereof. The care product composition could be provided by any suitable method in any suitable form for oral administration, such as in the form of a solution, syrup, a suspension, and also could be manufactured as a product such as a capsule, a beverage (e.g., a beauty beverage, a functional beverage, and a health beverage), but is not limited thereby.

As a form suitable for inhalation administration, the pharmaceutical composition and the care product composition of the present invention could optionally be aerosolized by any suitable approach to facilitate the entry of the pharmaceutical composition or the care product composition into the respiratory tract. For example, the pharmaceutical composition and the care product composition could be administered through a nebulizer, an aroma burner, an evaporative diffuser, an aroma lamp, an aroma diffuser, an essential oil candle, or an essential oil necklace, but is not limited thereby.

As a form for transdermal administration, the pharmaceutical composition of the present invention could also comprise any pharmaceutically acceptable carrier that will not adversely affect the desired effects of the active ingredient (i.e., Mentha essential oil). Examples of suitable carrier include, but are not limited to, water, mineral oil, propylene glycol, polyethylene oxide, liquid petrolatum, sorbitan monostearate, polysorbate 60. The pharmaceutical composition could be provided by any suitable method in any suitable form for transdermal administration, such as in a form of an emulsion (e.g., a massage emulsion), a cream (e.g., a massage cream), an oily product (e.g., a massage oil), a gel (e.g., as a hydrogel), a paste (e.g., a dispersing paste, an ointment), a douche, a spray, a patch (e.g., a microneedle patch), but is not limited thereby.

As a form for transdermal administration, the care product composition of the present invention could comprise any dermatologically acceptable carrier that will not adversely affect the desired effects of the active ingredient (i.e., Mentha essential oil). Examples of suitable carrier include, but are not limited to, water, mineral oil, propylene glycol, polyethylene oxide, liquid petrolatum, sorbitan monostearate, polysorbate 60. The care product composition can be provided by any suitable method in any suitable form for transdermal administration, such as in the form of an emulsion (e.g., a massage emulsion), a cream (e.g., a massage cream), a gel (e.g., a hydrogel), a solution (e.g., an essence and a lotion), a spray, but is not limited thereby.

Optionally, the pharmaceutical composition and the care product composition provided in accordance with the present invention could further comprise a suitable amount of additives, such as a toner, or a coloring agent for enhancing the visual perception of the pharmaceutical composition or the care product composition, and/or a buffer, a conservative, a preservative, an antibacterial agent, or an antifungal agent for improving the stability and storability of the pharmaceutical composition or the care product composition. Optionally, the pharmaceutical composition and the care product composition could further comprise one or more other active ingredients to further enhance the effects of the pharmaceutical composition or the care product composition, or to increase the application flexibility and adaptability of the preparation thus provided, as long as the other active ingredients do not adversely affect the desired effects of the active ingredients of the present invention (i.e., Mentha essential oil).

Depending on the needs, age, body weight, and health conditions of the subject as well as purpose(s), the pharmaceutical composition and the care product composition provided in accordance with the present invention could be administered at various administration frequencies, such as once a day, multiple times a day, or once every few days, etc. In addition, the concentration of the Mentha essential oil in the pharmaceutical composition and the care product composition could be adjusted depending on the requirements of practical application.

In the use of Mentha essential oil in helping reduce the production of free radical in accordance with the present invention, the Mentha essential oil could be provided as a care product composition and the administration type, administration route, administration form, administration frequency and uses of the care product composition are all in line with the above descriptions.

As described above, the present invention also provides a method for helping reduce the production of free radical, comprising administering to a subject in need an effective amount of Mentha essential oil, wherein “a subject in need” refers to a subject with excessive production of free radicals. In this method, the Mentha essential oil could be administered to the subject in need as a care product composition and the administration type, administration route, administration form, administration frequency and uses of the care product composition are also all in line with the above descriptions.

As described above, the present invention also provides a method for at least one of inhibiting obesity, preventing metabolic syndrome associated with obesity, treating neurodegenerative disease, preventing neurodegenerative disease, treating cardiovascular disease, preventing cardiovascular disease, treating type 2 diabetes, preventing type 2 diabetes, ameliorating sleep disturbance, regulating sleep, helping fall asleep, inhibiting inflammation, treating cancer, and preventing cancer, comprising administering to a subject in need an effective amount of Mentha essential oil, wherein “a subject in need” refers to a subject having obesity phenomena, suffering from neurodegenerative disease, suffering from cardiovascular disease, suffering from type 2 diabetes, having sleep disturbance, having inflammation phenomena, and/or suffering from cancer, or a subject with high-risk in the above diseases. In this method, the Mentha essential oil could be administered to the subject in need as a pharmaceutical composition and the administration type, administration route, administration form, administration frequency and uses of the pharmaceutical composition are also all in line with the above descriptions.

The present invention also provides a method for at least one of increasing expression of SOD1 gene, increasing expression of CAT gene, increasing expression of UCP2 gene, increasing expression of CRY gene, increasing expression of PER2 gene, decreasing expression of IL-8 gene, and decreasing expression of IL-1β gene, comprising administering to a subject in need an effective amount of Mentha essential oil, wherein “a subject in need” refers to a subject whose SOD1 gene, CAT gene, UCP2 gene, CRY gene, and/or PER2 gene is deleted, mutated, or low-expressed, or a subject whose IL-8 gene and/or IL-1β gene is overexpressed. In this method, the Mentha essential oil could be administered to the subject in need as a pharmaceutical composition and the administration type, administration route, administration form, administration frequency and uses of the pharmaceutical composition are also all in line with the above descriptions.

The present invention will be further illustrated in detail with specific examples as follows. However, the following examples are provided only for illustrating the present invention and the scope of the present invention is not limited thereby. The scope of the present invention will be indicated in the appended claims.

### EXAMPLES

### Example 1: Effects of Mentha Essential Oil on Regulating the Expressions of SOD1 Gene, CAT Gene, UCP2 Gene, CRY Gene, PER2 Gene, IL-8 Gene and/or IL-1β Gene

### (1-1) Collection of Blood Samples

The following two-week trial was conducted on five people (five subjects) to understand the effects of Mentha essential oil on human body. During the trial, each subject wore an essential oil necklace containing Mentha essential oil (purchased from Agronatura COOP.R.L; product number: UK2122) for 8 hours, and the essential oil necklace was added with 20 μL of Mentha essential oil at nine o'clock every morning to ensure that the amount of essential oil in the necklace was sufficient. And, 6 mL blood sample was collected from each subject prior to the trial (i.e., prior to starting wearing the essential oil necklace containing Mentha essential oil; hereinafter referred to as “W0” group), after starting the trial for one week (i.e., wearing the essential oil necklace containing Mentha essential oil eight hours a day and for one week; hereinafter referred to as “W1” group), and after starting the trial for two weeks (i.e., wearing the essential oil necklace containing Mentha essential oil eight hours a day and for two weeks; hereinafter referred to as “W2” group), respectively, by using blood collection tubes that contained ethylenediaminetetraacetic acid (EDTA) (purchased from VACUETTE®, product number: 456036). The blood samples thus obtained were used for the following experiments and analysis.

### (1-2) Isolation of Peripheral Blood Mononuclear Cells (PBMC)

The blood samples of “W0” group, “W1” group and “W2” group obtained from Example (1-1) were individually subjected to the following steps to isolate PBMCs from the blood samples of each group:

1. Injecting each blood sample to a 15 mL centrifuge tube, and subjecting the centrifuge tube to a centrifugation at 300 g for 15 minutes (the centrifugation was stopped by natural deceleration);
2. Removing the upper blood plasma layer, taking out 2 mL of buffy coat from the remnant and putting the same to another 15 mL centrifuge tube, and then adding thereinto 2 mL of phosphate buffered saline (PBS) and mixing evenly to provide a diluted buffy coat;
3. Tilting another 15 mL centrifuge tube loaded with 3 mL of Ficoll-Plague Plus (purchased from Sigma company, product number: GE17-1440-03) for about 45 degrees, then slowly adding the diluted buffy coat provided by step 2 into the centrifuge tube and carefully returning the centrifuge tube back to the vertical situation, so that the buffy coat and Ficoll-Plague Plus were layered (not mixed), and then, subjecting the centrifuge tube to a centrifugation at 400 g for 40 minutes (the centrifugation was stopped by natural deceleration);
4. Removing the upper liquid layer, taking out 2 mL to 3 mL of the middle layer (i.e., mononuclear cell layer) and putting the same to a new 15 mL centrifuge tube, then, washing the cells with PBS three to five times, and then, subjecting the washed cells to a centrifugation at 300 g for 10 minutes; and
5. Removing the remaining PBS to provide the precipitate, i.e., the PBMCs of “W0” group, “W1” group, or “W2” group.

### (1-3) Gene Expression Analysis

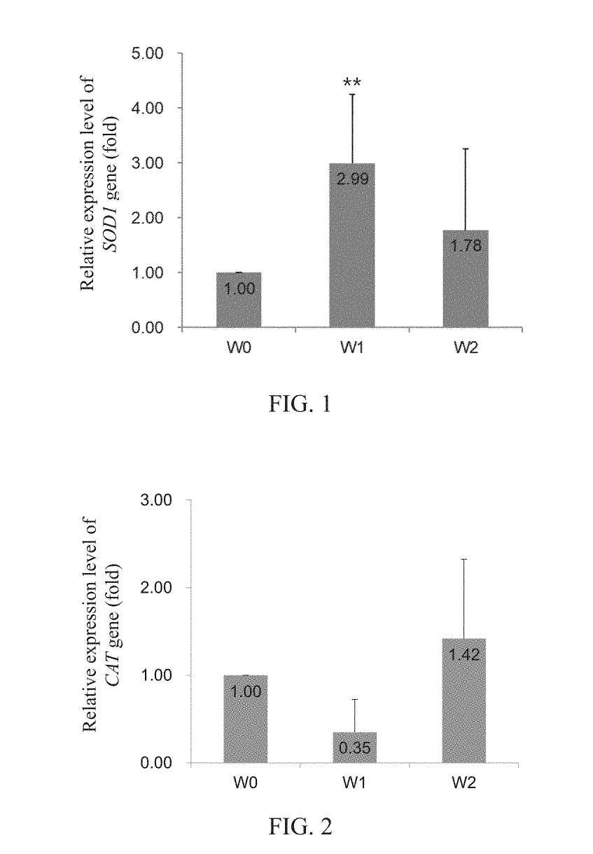

The PBMCs of “W0” group, “W1” group and “W2” group obtained from Example (1-2) were lysed by an RNA lysis buffer (RB buffer; purchased from Geneaid company). Thereafter, the PBMC lysate of each group was subjected to an RNA extraction with an RNA extraction kit (purchased from Geneaid company), and then the RNA was transcribed into cDNA with a reverse transcriptase (SuperScript® III Reverse Transcriptase; purchased from Invitrogen company). Then, the cDNA of each group was subjected to a quantitative polymerase chain reaction (qPCR) by using an ABI Step One Plus system and a KAPA SYBR FAST qPCR kit to determine the expression levels of SOD1, CAT, UCP2, CRY, PER2, IL-8 and IL-1β genes in the cells of each group. Finally, the data was analyzed by T.TEST of Excel (one tailed Student's t-test), and the analysis result of “W0” group was used as a basis (i.e., the gene expression of “W0” group was set as 1-fold) to calculate the relative gene expression level of “W1” group and that of “W2” group. The results are shown in FIGS. 1 to 7.

As shown in FIG. 1, as compared to “W0” group, the expression levels of SOD1 gene in the cells of “W1” group and “W2” group significantly increased. These results indicate that Mentha essential oil can effectively increase the expression level of SOD1 gene, and thus, can be used for treating amyotrophic lateral sclerosis, preventing amyotrophic lateral sclerosis and/or helping reduce the production of free radical.

As shown in FIG. 2, as compared to “W0” group, the expression levels of CAT gene in the cells of “W2” group significantly increased. These results indicate that Mentha essential oil can effectively increase the expression level of CAT gene, and thus, can be used for treating type 2 diabetes, preventing type 2 diabetes and/or helping reduce the production of free radical.

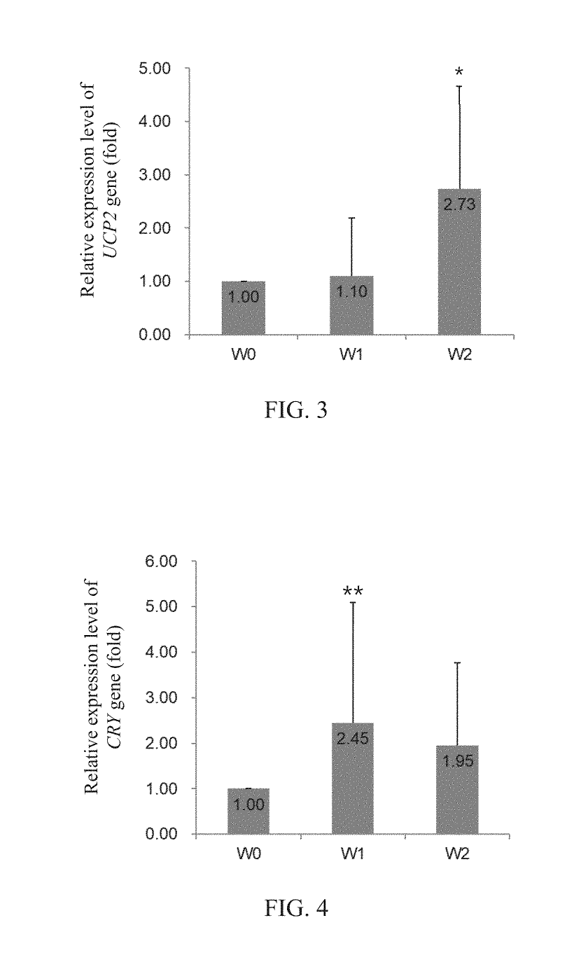

As shown in FIG. 3, as compared to “W0” group, the expression levels of UCP2 gene in the cells of “W1” group and “W2” group significantly increased. These results indicate that Mentha essential oil can effectively increase the expression level of UCP2 gene, and thus, can be used for inhibiting obesity, preventing metabolic syndrome associated with obesity, making body fat be difficult to form, treating neurodegenerative disease, preventing neurodegenerative disease, treating cardiovascular disease and/or preventing cardiovascular disease.

As shown in FIG. 4, as compared to “W0” group, the expression levels of CRY gene in the cells of “W1” group and “W2” group significantly increased. These results indicate that Mentha essential oil can effectively increase the expression level of CRY gene, and thus, can be used for ameliorating sleep disturbance, regulating sleep, helping fall asleep and/or treating atherosclerosis.

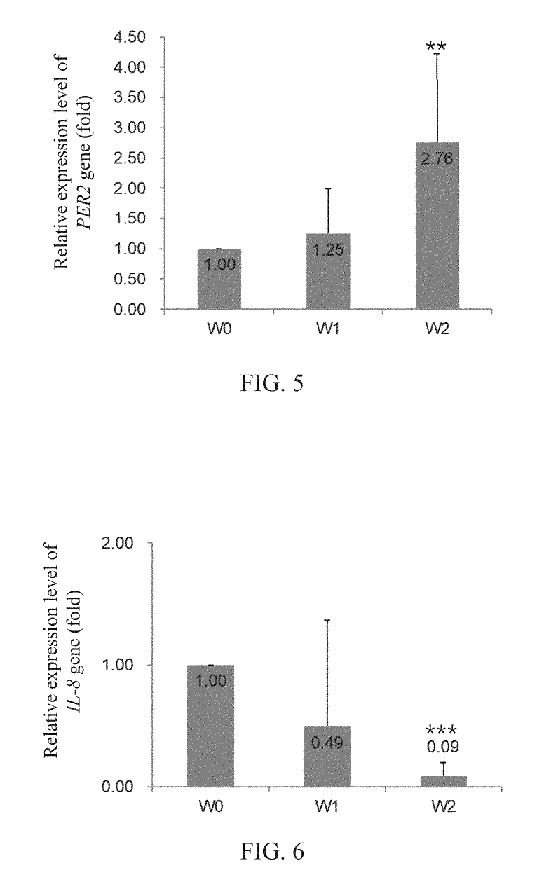

As shown in FIG. 5, as compared to “W0” group, the expression levels of PER2 gene in the cells of “W1” group and “W2” group significantly increased. These results indicate that Mentha essential oil can effectively increase the expression level of PER2 gene, and thus, can be used for ameliorating sleep disturbance, regulating sleep and/or helping fall asleep.

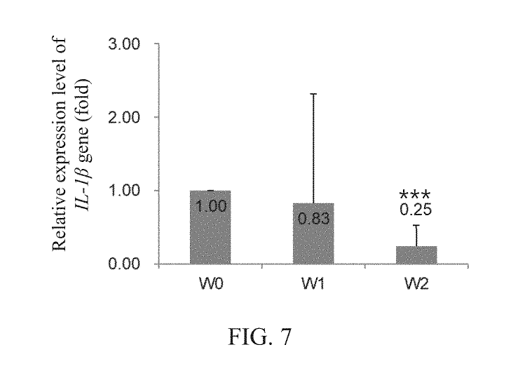

As shown in FIGS. 6 and 7, as compared to “W0” group, the expression levels of IL-8 and IL-1β genes in the cells of “W1” group and “W2” group all significantly decreased. These results indicate that Mentha essential oil can effectively decrease the expression level of IL-8 and IL-1β genes, and thus, can be used for inhibiting inflammation, treating cancer and/or preventing cancer.

What is claimed is:

1. A method for at least one of inhibiting obesity, preventing metabolic syndrome associated with obesity, treating neurodegenerative disease, preventing neurodegenerative disease, treating cardiovascular disease, preventing cardiovascular disease, treating type 2 diabetes, preventing type 2 diabetes, ameliorating sleep disturbance, regulating sleep, helping fall asleep, inhibiting inflammation, treating cancer and preventing cancer, comprising administering to a subject in need an effective amount of Mentha essential oil.

2. The method as claimed in claim 1, which is for at least one of inhibiting obesity, preventing metabolic syndrome associated with obesity, treating neurodegenerative disease, preventing neurodegenerative disease, treating cardiovascular disease and preventing cardiovascular disease.

3. The method as claimed in claim 2, which is for making body fat be difficult to form.

4. The method as claimed in claim 1, which is for at least one of treating amyotrophic lateral sclerosis, preventing amyotrophic lateral sclerosis, treating type 2 diabetes and preventing type 2 diabetes.

5. The method as claimed in claim 1, which is for at least one of ameliorating sleep disturbance, regulating sleep, helping fall asleep and treating atherosclerosis.

6. The method as claimed in claim 5, which is for at least one of ameliorating sleep disturbance, regulating sleep and helping fall asleep.

7. The method as claimed in claim 1, which is for at least one of inhibiting inflammation, treating cancer and preventing cancer.

8. The method as claimed in claim 7, wherein the cancer is as least one of lung cancer, melanoma and colon cancer.

9. The method as claimed in claim 1, wherein the Mentha essential oil is administered to the subject by at least one of oral administration, inhalation administration and transdermal administration.

10. The method as claimed in claim 2, wherein the Mentha essential oil is administered to the subject by at least one of oral administration, inhalation administration and transdermal administration.

11. The method as claimed in claim 3, wherein the Mentha essential oil is administered to the subject by at least one of oral administration, inhalation administration and transdermal administration.

12. The method as claimed in claim 4, wherein the Mentha essential oil is administered to the subject by at least one of oral administration, inhalation administration and transdermal administration.

13. The method as claimed in claim 5, wherein the Mentha essential oil is administered to the subject by at least one of oral administration, inhalation administration and transdermal administration.

14. The method as claimed in claim 6, wherein the Mentha essential oil is administered to the subject by at least one of oral administration, inhalation administration and transdermal administration.

15. The method as claimed in claim 7, wherein the Mentha essential oil is administered to the subject by at least one of oral administration, inhalation administration and transdermal administration.

16. The method as claimed in claim 8, wherein the Mentha essential oil is administered to the subject by at least one of oral administration, inhalation administration and transdermal administration.

17. A method for helping reduce the production of free radical, comprising administering to a subject in need an effective amount of Mentha essential oil.

18. The method as claimed in claim 17, wherein the Mentha essential oil is administered to the subject by at least one of oral administration, inhalation administration and transdermal administration.

19. A method for at least one of increasing expression of SOD1 gene, increasing expression of CAT gene, increasing expression of UCP2 gene, increasing expression of CRY gene, increasing expression of PER2 gene, decreasing expression of IL-8 gene and decreasing expression of IL-1β gene, comprising administering to a subject in need an effective amount of Mentha essential oil.

20. The method as claimed in claim 19, wherein the Mentha essential oil is administered to the subject by at least one of oral administration, inhalation administration and transdermal administration.
